(19) United States
(12) Patent Application Publication
(10) Pub. No.: US 2026/0283697 A1
(43) Pub. Date: Sep. 24, 2026

(54) MULTI-IMAGE INFORMATION FUSION METHOD FOR TISSUE CUTTING PATH PLANNING, SYSTEM, MEDIUM, AND ELECTRONIC DEVICE

(71) Applicant: HEALINNO (BEIJING) MEDICAL TECHNOLOGY CO., LTD., Beijing (CN)

(72) Inventors: Yilun SHI, Beijing (CN); Ce SHI, Beijing (CN); Hao LI, Beijing (CN); Wenbo CHEN, Beijing (CN); Jing ZHAO, Beijing (CN)

(21) Appl. No.: 19/646,779

(22) Filed: Apr. 14, 2026

Related U.S. Application Data
(62) Division of application No. 18/660,273, filed on May 10, 2024.
Application No. 18/660,273 is a continuation of application No. PCT/CN2022/131742, filed on Nov. 14, 2022.

(30) Foreign Application Priority Data
Nov. 12, 2021 (CN) 202111339201.0

(51) Int. Cl. A61B 34/10; A61B 1/00; A61B 8/00; A61B 18/00; A61B 34/20

(52) U.S. Cl. CPC A61B 34/10; A61B 1/00009; A61B 1/00057; A61B 8/483; A61B 8/523; A61B 8/5261; A61B 18/00; A61B 2018/00577; A61B 2018/00982; A61B 2034/107; A61B 2034/2063; A61B 2034/2065

(57) ABSTRACT

A multi-image information fusion method for tissue cutting path planning, comprising: acquiring position information of a target tissue working area calibrated in an endoscopic image, and converting the position information of the target tissue working area into coordinate information in a standard coordinate system, so as to obtain an endoscopic image in the standard coordinate system; acquiring a three-dimensional ultrasound image of a target tissue; and extracting two-dimensional slice image contour position information of the three-dimensional ultrasound image, and converting the two-dimensional slice image contour position information into coordinate information in the standard coordinate system, so as to obtain an ultrasound image in the standard coordinate system. An endoscopic apparatus comprises an endoscope and a position feedback apparatus, and the tissue cutting path planning is performed on the basis of the endoscopic image in the standard coordinate system and the ultrasound image in the standard coordinate system.

### RELATED APPLICATIONS

The present application is a divisional of U.S. application Ser. No. 18/660,273, filed on May 10, 2024, which is a continuation of International Application No. PCT/CN2022/131742, filed on Nov. 14, 2022, which claims priority to Chinese Patent Application No. 202111339201.0, filed on Nov. 12, 2021, and entitled “MULTI-IMAGE INFORMATION FUSION METHOD FOR TISSUE CUTTING PATH PLANNING, SYSTEM, MEDIUM, AND ELECTRONIC DEVICE,” the entire disclosures of the foregoing applications are incorporated herein by reference.

### BACKGROUND

### 1. Technical Field

This application relates to the field of medical devices, and in particular, to a multi-image information fusion method and system for automatic tissue cutting path planning.

### 2. Background Information

For the treatment of hyperplastic or cancerous tissues, such as benign prostatic hyperplasia (BPH) and prostate cancer, in addition to drug therapy, conventional surgical resection or partial resection surgery is commonly used for a very long time. This method generally relies on open incisions, and has disadvantages such as strong invasiveness, large trauma, and a long recovery period. Post-minimally invasive resection therapy has been widely applied in this field. For example, energy such as laser, water jet, and optical fiber is used to resect and/or burn a lesion or hyperplastic tissue in prostate and other tissues as a fluid flow, which generally enters through the urethra without the need for an open incision, and has the advantage of minimal trauma.

When energy is used to resect tissues such as the prostate, it is necessary to plan a tissue cutting path in advance. The accuracy of cutting path planning not only affects the efficiency of surgery, but also relates to the safety and reliability of surgery. The planning of the cutting path in the prior art is usually performed by means of an ultrasound image. The ultrasound image of the target tissue is acquired by an ultrasonic probe, and a reference structure of the ultrasonic probe is provided. After adjustments, the ultrasound image aligned with the reference structure is acquired. Doctors manually input contour parameters by reading image information and plan the cutting path according to the input parameter information. For sensitive or key parts (such as seminal colliculus), manual marking by doctors is required. It can be seen that the planning of the existing cutting path has a low automation level, and requires a large amount of manual participation. Given the objective effects of image quality and manual handling errors, this cutting path planning method in the prior art not only requires a large amount of manual participation, which makes the operation complex and increases the workload of the medical staff, but also has low accuracy, making it difficult to precisely acquire the contour position, particularly the accurate position of the seminal colliculus or the bladder neck, which leads to lower precision of surgical cutting, incomplete cutting, and lower safety.

In terms of the current state of technological development, there are still many technical obstacles to be overcome to achieve truly automated planning of a tissue cutting path, such as how to achieve the automatic and precise marking of sensitive parts, critical parts, and cutting starting and ending positions on a three-dimensional ultrasound image, and how to automatically mark the contour information of the target tissues.

### BRIEF SUMMARY

It is an object of this application to provide a multi-image information fusion method and system for automatic tissue cutting path planning. By providing an endoscopic apparatus with a position feedback apparatus, and fusing image information acquired by the endoscopic apparatus with three-dimensional ultrasound image information in the same coordinate system, the image information acquired by the endoscope can be directly calculated with the three-dimensional ultrasound image information, so as to automatically and precisely mark sensitive parts, key parts, cutting starting and ending positions, etc. on the three-dimensional ultrasound image, providing technical support for achieving truly automatic planning of a cutting path.

To achieve the above invention object, this application employs the following technical solutions.

A multi-image information fusion method for automatic tissue cutting path planning according to this application includes the following steps:
acquiring position information of a target tissue working area calibrated in an endoscopic image photographed by an endoscopic apparatus, and converting the position information of the target tissue working area into coordinate information in a standard coordinate system, so as to obtain an endoscopic image in the standard coordinate system;
acquiring a three-dimensional ultrasound image of a target tissue; and
extracting two-dimensional slice image contour position information of the three-dimensional ultrasound image, and converting the two-dimensional slice image contour position information into coordinate information in the standard coordinate system, so as to obtain an ultrasound image in the standard coordinate system, wherein
the endoscopic apparatus includes an endoscope and a position feedback apparatus, and
the tissue cutting path planning is performed on the basis of the endoscopic image in the standard coordinate system and the ultrasound image in the standard coordinate system.

In the multi-image information fusion method for automatic tissue cutting path planning according to this application, preferably, the method further includes the following steps: selecting a coordinate system where a fixed reference component is located as the standard coordinate system, and converting the acquired position information into the coordinate information in the standard coordinate system by a coordinate transformation matrix.

Preferably, the coordinate transformation matrix is obtained by relative calibration.

Preferably, the step of acquiring the position information of the target tissue working area calibrated by the endoscopic apparatus includes: acquiring cutting starting position information and cutting ending position information of an ablation tool calibrated by the endoscopic apparatus.

Preferably, the steps of acquiring the cutting starting position information of the ablation tool include: moving the endoscopic apparatus to a position where a urethral orifice is observed through a bladder neck, calibrating the position as the cutting starting position, and automatically acquiring, by the position feedback apparatus, the cutting starting position information.

Preferably, the step of acquiring the cutting ending position information of the ablation tool includes: moving the endoscopic apparatus to a seminal colliculus position, calibrating the position as the cutting ending position, and automatically acquiring, by the position feedback apparatus, the cutting ending position information.

Preferably, the step of extracting the two-dimensional slice image contour position information includes: extracting ablation tool contour position information and target tissue contour position information.

This application further provides a multi-image information fusion system for automatic tissue cutting path planning, including:
a motion control apparatus, wherein the motion control apparatus includes a fixed reference component, and a first motion control component and a second motion control component that are connected to the fixed reference component;
an ablation tool module, wherein the ablation tool module includes an ablation tool and an endoscope with a position feedback apparatus, the ablation tool module can acquire position information of a target tissue working area, and both the endoscope and the ablation tool are connected to the first motion control component;
a three-dimensional ultrasound imaging module, wherein the three-dimensional ultrasound module includes an ultrasonic probe, the ultrasonic probe can be used for acquiring three-dimensional ultrasound image information of a target tissue, and the ultrasonic probe is connected to the second motion control component; and
a processor, wherein the processor can extract two-dimensional slice image contour position information of the three-dimensional ultrasound image, and fuse the acquired position information of the target tissue working area and the two-dimensional slice image contour position information of the target tissue into the same standard coordinate system.

In the multi-image information fusion system for automatic tissue cutting path planning according to this application, preferably, both the endoscope and the ablation tool are connected to the first motion control component through a first adapter; and the ultrasonic probe is connected to the second motion control component through a second adapter.

Preferably, the first motion control component and/or the second motion control component is a mechanical arm or a support provided with a position feedback apparatus.

Preferably, the position feedback apparatus is an encoder.

Preferably, the standard coordinate system is a coordinate system where the fixed reference component is located.

This application further provides a computer-readable storage medium storing a computer program thereon, characterized in that, the program, when executed by a processor, implements the method according to any one of the embodiments of this application.

This application further provides an electronic device, including a memory, a processor, and a computer program that is stored in the memory and can be run in the processor, and the processor implements the method according to any one of the embodiments of this application when executing the computer program.

This application has the following beneficial effect: In the technical solutions of this application, the endoscopic apparatus provided with the position feedback apparatus is introduced, so that the image information of the tissue to be cut can be acquired through the endoscopic apparatus, and by means of the multi-image information fusion method according to this application, the image information obtained by the endoscope and the three-dimensional ultrasound image information are fused into the same standard coordinate system, which helps to achieve automatic and precise calibration of sensitive position areas that need to be avoided during cutting, such as the seminal colliculus position, as well as the cutting starting and ending positions, and helps to solve the problems of excessive manual participation and low accuracy in the prior art. At the same time, it provides technical support for truly achieving automatic planning of a cutting path.

### BRIEF DESCRIPTION OF THE DRAWINGS

The accompanying drawings described herein are used to provide a further understanding of this application, and form part of this application. Exemplary embodiments of this application and descriptions thereof are used to explain this application, and do not constitute any inappropriate limitation to this application. In the drawings:

FIG. 1 is a schematic diagram of a specific embodiment of a multi-image information fusion system for automatic tissue cutting path planning according to this application;

FIG. 2 is a schematic flowchart of a multi-image information fusion method for automatic tissue cutting path planning according to this application;

FIG. 3 is a schematic diagram of the endoscopic field of view according to this application;

FIG. 4 is a schematic flowchart of an embodiment of employing a two-dimensional image endoscope to acquire position information according to this application;

FIG. 5 is a schematic flowchart of an embodiment of employing a stereo vision endoscope to acquire position information according to this application; and

FIG. 6 is a diagram for illustrating a method of tissue resection path planning on the basis of an endoscopic image in a standard coordinate system and an ultrasound image in the standard coordinate system according to this application.

### DETAILED DESCRIPTION OF THE DRAWINGS AND THE PRESENTLY PREFERRED EMBODIMENTS

To make the objectives, technical solutions, and advantages of this application clearer, a clear and complete description of the technical solutions of this application will be given below, in conjunction with the specific embodiments and the corresponding drawings of this application. Apparently, the embodiments described below are merely some, but not all, of the embodiments of this application. Although many technical details are described in detail in the specific embodiments section of this application, it should be noted that these details do not constitute a limitation on the scope of protection of this application. Any improvements or changes made by those of ordinary skill in the art on the basis of the technical solutions disclosed in this application without any inventive efforts are also within the scope of protection of this application.

It should be noted that although this application is based on the background of prostate tissue resection, a method and system for fusion of multi-image information such as endoscopic image information and ultrasound image information in the automatic tissue resection path planning process in this application are not limited to prostate tissue, but can also be used to process any other similar human tissue organs, such as kidneys, liver, skin, muscles, glands, esophagus, throat, and intestine. Under the basic methods, objectives, and spirit of this application, those skilled in the art can make adaptive adjustments to the method and system according to the differences of the target tissue, which is also within the scope of protection of this application.

The term “ablation tool” used in this application has the following meanings: it refers to a tool that cuts and burns tissue by means of energy (such as water jets, lasers, and electricity), so that the target tissue or focus tissue is ablated (i.e., reduced in volume).

The technical solutions provided by the embodiments of this application will be described in detail below in conjunction with the drawings.

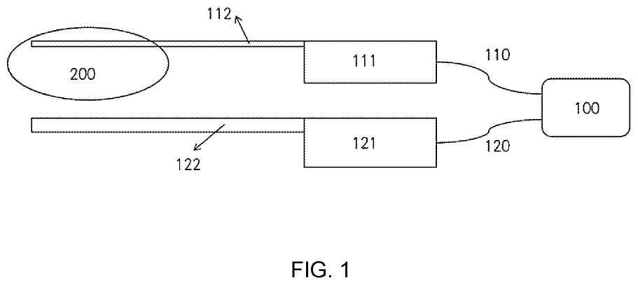

As shown in FIG. 1, a multi-image information fusion system for automatic tissue cutting path planning according to this application includes a motion control apparatus, an ablation tool module, a three-dimensional ultrasound imaging module and a processor. The motion control apparatus includes a fixed base 100 as a fixed reference component, a first mechanical arm 110 and a second mechanical arm 120 for rotation-fit connection with the fixed base 100, wherein ends of the first mechanical arm 110 and the second mechanical arm 120 are each provided with an encoder, or other similar position feedback apparatuses or positioning apparatuses that can be used to transmit the positions of the first mechanical arm and the second mechanical arm. The first mechanical arm 110 and/or second mechanical arm 120 may be the same or different, and those skilled in the art can select as required. For example, 6-axis or 7-axis mechanical arms may be selected, both may be active mechanical arms or passive mechanical arms, or one is an active mechanical arm and the other is a passive mechanical arm. Moreover, in some embodiments, a first mechanical arm 110 and/or a second mechanical arm 120 can also be replaced with a rotatable support.

The fixed base 100 is mainly used as a fixed reference, without any restrictions on its structure, and the coordinate system with the fixed base 100 is used as a standard coordinate system. The fixed base 100 is provided with one or more processors (CPU) built-in or externally connected, which are used to complete data processing and control work, such as motion control, coordinate transformation, and image extraction processing.

The ablation tool module includes an ablation tool, as well as an endoscope with an encoder (not shown in the figure), wherein the encoder can also be replaced with other apparatus that is used to feed back the position information. The ablation tool can utilize energy to cut the focus tissue. The ablation tool can use water flow, laser, optical fiber, electrodes, etc. as energy sources to cut and burn the focus tissue, thereby achieving the resection of the focus tissue. The ablation tool and the endoscopic apparatus are integrated into a sheath 112. Rear ends of the ablation tool and the endoscopic apparatus extend out of the sheath 112 to be inserted and fitted with a first adapter 111 fixedly provided at the front end of the first mechanical arm 110, so that the first mechanical arm 110 drives the ablation tool and endoscopic apparatus to move forward/backward/rotatably under the control of the processor. The sheath 112 is in the form of a slender tubular. The sheath 112 can extend into the prostate 200 along the urethra, and the position information of the focus tissue to be cut is acquired through the endoscope and the endoscope encoder.

The three-dimensional ultrasound imaging module includes a slender tubular ultrasonic probe 122. The rear end of the ultrasonic probe 122 is inserted and fitted with a second adapter 121 fixedly provided at the front end of a second mechanical arm 120, and the processor controls the ultrasonic probe to move forward/backward/rotatably through the second mechanical arm 120 and the second adapter 121. During the process that the ultrasonic probe 122 moves forward at a particular speed under the control of the second mechanical arm 120, the processor captures two-dimensional ultrasonic slice images at a specific step size, extracts two-dimensional slice image contour position information and records the information. The processor converts prostate focus tissue position information and two-dimensional slice image position information into coordinates in a coordinate system where the fixed base 100 is located through coordinate transformation matrices respectively, so that the prostate focus tissue position information and the two-dimensional slice image position information are located in the same coordinate system, and then the path planning steps can be performed. The specific steps of specific coordinate transformation are detailed below.

It should be noted that, in some other embodiments, the first adapter 111 and the second adapter 121 are not necessary, and other connecting components may alternatively be selected.

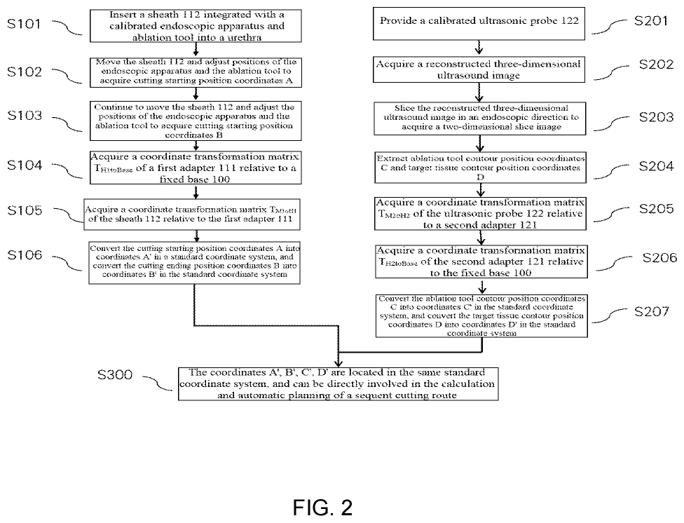

FIG. 2 shows a multi-image information fusion method for automatic tissue cutting path planning according to this application, including the following steps.

Step S101: An endoscopic apparatus, an ablation tool, and a sheath 112 integrated with the endoscopic apparatus and the ablation tool are calibrated, and the sheath 112 integrated with the calibrated endoscopic apparatus and ablation tool is inserted into a urethra.

Step S102: A processor controls the sheath 112 to move forward along the urethra at a certain speed through a first mechanical arm 110, and adjusts the positions of the endoscopic apparatus and the ablation tool during this process, and an endoscope encoder feeds back endoscope position information to the processor in real time; when the endoscopic apparatus is moved to a position of a urethral orifice that can be observed through a bladder neck, the endoscope encoder feeds back coordinate information of this position to the processor, and the processor calibrates this position as a cutting starting position, and extracts cutting starting position coordinates A (xₐ₁,yₐ₁,zₐ₁).

Step S103: The first mechanical arm 110 drives the sheath 112 to continue to move forward under the control of the processor; when the endoscope reaches a position past the seminal colliculus or reaches an equivalent position as determined by the processor, the endoscope encoder feeds back coordinate information of this position to the processor, and the processor calibrates this position as a cutting ending position, and extracts cutting ending position coordinates B (xₐ₂,yₐ₂,zₐ₂). In this process, data information on a plurality of positions of starting and ending in the seminal colliculus determined from the endoscopic image when the endoscope reaches near the seminal colliculus may be recorded, thus providing accurate position information of the seminal colliculus part for the planning process.

Step S104: With a coordinate system where the fixed base 100 is located as a standard coordinate system, a coordinate transformation matrix T_{H1toBase} of a first adapter 111 relative to fixed base 100 is acquired by a built-in program of the process.

Step S105: Similar to step S104, a coordinate transformation matrix T_{M1oH1} of the sheath 112 relative to the first adapter 111 is acquired by the built-in program of the process.

Step S106: The cutting starting position coordinates A are converted into coordinates A′ in the standard coordinate system, and the cutting ending position coordinates B are converted into coordinates B′ in the standard coordinate system, according to the following formula, wherein

$${A}^{′} = {T}_{M 1 oH 1} \cdot {T}_{H 1 toBase} \cdot A = ( {x}_{c 1} , {y}_{c 1} , {z}_{c 1} )$$

$${B}^{′} = {T}_{M 1 oH 1} \cdot {T}_{H 1 toBase} \cdot B = ( {x}_{c 2} , {y}_{c 2} , {z}_{c 2} )$$

Step S201: An ultrasonic probe 122 is calibrated, and the calibrated ultrasonic probe 122 is placed in a scanning starting position.

Step S202: The processor controls the ultrasonic probe 122 to move to the ending position along a preset path through the second mechanical arm 120, and all ultrasound image data sequences of the target tissue acquired during the motion process are fed back to the processor. The processor performs three-dimensional reconstruction according to the acquired ultrasound image data sequences, so as to form an overall three-dimensional ultrasound image of the target tissue. In this embodiment, the target tissue is a prostate tissue.

Step S203: During the process of moving the ultrasonic probe 122 along the presetting path from the starting position to the ending position, the processor captures a two-dimensional slice image along the axial motion direction of the endoscope in a particular step, and adjusts the ultrasonic probe 122 to be parallel to the ablation tool while slicing, ensuring that the ablation tool can be clearly observed within the two-dimensional slice image.

Step S204: Contour extraction is performed on the two-dimensional slice image of the target tissue prostate to acquire a contour position coordinate C (x_{b1},y_{b1},z_{b1}) of the ablation tool, and a contour position coordinate D(x_{b2},y_{b2},z_{b2}) of the prostate.

Step S205: With the coordinate system where the fixed base 100 is located as the standard coordinate system, a coordinate transformation matrix T_{H2toBase} of the second adapter 121 relative to fixed base 100 is acquired by the process built-in program.

Step: S206: Similar to Step S205, a coordinate transformation matrix T_{M2oH2} of the ultrasonic probe 122 relative to the second adapter 121 is acquired by the process built-in program.

Step S207: Contour position coordinates C of the ablation tool are converted into coordinates C′ in the standard coordinate system, and prostate contour position coordinates D of the target tissue are converted into coordinates D′ in the standard coordinate system, wherein

$${C}^{′} = {T}_{M 2 oH 2} \cdot {T}_{H 2 toBase} \cdot C = ( {x}_{c 3} , {y}_{c 3} , {z}_{c 3} )$$

$${D}^{′} = {T}_{M 2 oH 2} \cdot {T}_{H 2 toBase} \cdot D = ( {x}_{c 4} , {y}_{c 4} , {z}_{c 4} )$$

Step S300: After the above steps, the coordinate conversion between the position information calibrated by the endoscopic apparatus and the three-dimensional ultrasound image information in the same standard coordinate system is completed. At this point, the cutting starting position coordinates A', the cutting ending position coordinates B′, the contour position coordinates C′ of the ablation tool and the prostate contour position coordinates D′ of the target tissue, and the like in the same standard coordinate system can directly undergo position coordinate calculation to acquire their distance and path information directly, thus providing technical support for a smooth implementation of the subsequent automatic cutting path planning.

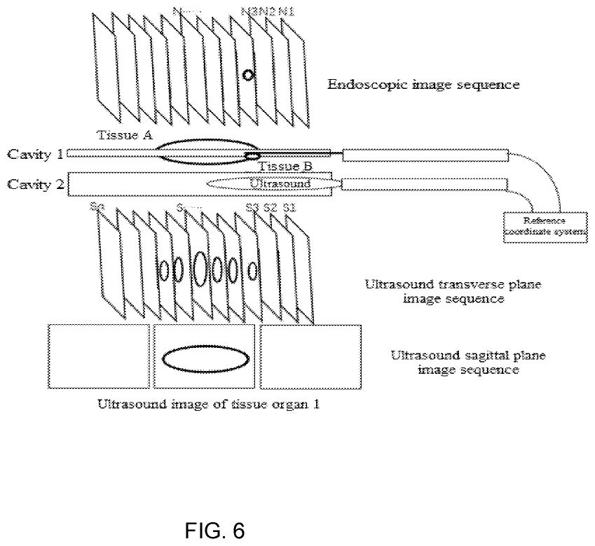

Next, with reference to FIG. 6, a method of tissue resection path planning on the basis of the endoscopic image in the standard coordinate system and the ultrasound image in the standard coordinate system is illustrated.

As shown in FIG. 6, taking water jet prostate resection surgery as an example, planning is usually completed on ultrasound images (sagittal and cross-sectional images). A resection surgery planning requires resection of a tissue organ A (prostate), while avoiding a tissue organ B (such as seminal colliculus). The image of the tissue organ A (prostate) can be obtained on biplane ultrasound images, but the ultrasound image is affected by resolution precision, noise, etc., and the accurate position of the tissue organ B (such as seminal colliculus) cannot be obtained. At this point, it is necessary to obtain the accurate position of the tissue organ B by the endoscopic observation to provide accurate information for the resection surgery planning. Therefore, it is necessary to fuse the accurate position of the tissue organ B observed by the endoscope and the accurate position of the tissue organ A observed by ultrasound through a reference coordinate system constructed by the executing mechanism to achieve the accurate position of the tissue organ B on the ultrasound image, providing information for completing subsequent resection surgery planning on the ultrasound image.

It should be noted that the sequence of above steps is only an illustration for the purpose of clearly illustrating this embodiment and does not constitute a limitation on the sequence of processing steps. In fact, the above steps can be completed in different orders, and those skilled in the art can make adjustments as needed. Some of steps may be added and/or deleted, and some steps may further include several sub-steps, and more conventional processing steps will not be repeated herein. If it is favorable to process, some of the above steps can also be repeated.

In some embodiments, the coordinate transformation matrix is obtained by relative calibration. For example, T_{M1oH1} and T_{H1toBase} are obtained based on calibration of a fixed adapter connected to a water jet module, a fixed structure of the adapter, and a fixed reference, and T_{M2oH2} and T_{H2toBase} are obtained based on calibration of the ultrasound images and the fixed adapter of an ultrasound module, the fixed structure of the adapter, and the fixed reference. The number of coordinate transformation matrices is determined by how many relative motion components are involved, and in some embodiments, only the coordinate transformation matrices T_{H1toBase} and T_{H2toBase} may be contained. In some embodiments, coordinate transformation matrices T_{M1oH1}, T_{H1toBase}, T_{M2oH2} and T_{H2toBase} may be 3×3 or 4×4 rotation matrices, or the like. In some other embodiments, the coordinate transformation matrix may also employ other matrix forms, which may be determined by those skilled in the art as needed, provided that spatial transformation of the coordinates can be implemented. A selected specific program may be pre-built into the processor, and a coordinate transformation matrix may be determined based on the position information fed back by the encoder and based on preset program instructions for the calculation of the subsequent coordinate transformation.

In some other embodiments, the image information acquired by using the endoscopic apparatus includes, in addition to the cutting starting position coordinates and cutting ending position coordinates, other coordinate information of positions whose resection is undesired or coordinate information of sensitive positions that needs to be avoided, etc. Those skilled in the art can make specific selections according to the characteristics of the target tissue and the focus tissue.

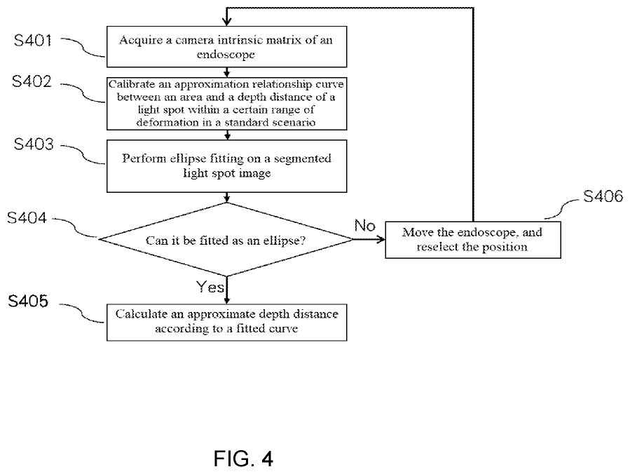

In some embodiments, a two-dimensional image endoscope is employed to acquire the target position information, and an acquisition method 400, as shown in FIG. 4, includes the following specific steps.

Step S401: An endoscope position is selected, a camera intrinsic matrix of an endoscope is acquired, and initial position coordinate information of the endoscope is acquired.

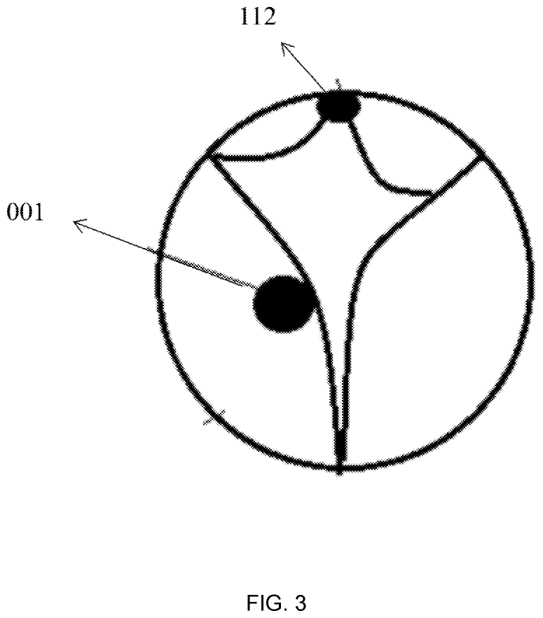

Step S402: An area and a depth distance of a light spot 001 within a certain range of deformation are measured in a standard scenario to acquire a fitting relationship curve between the area and the depth of the light spot. The schematic diagram of the endoscopic field of view is shown in FIG. 3.

Step S403: A light spot image is segmented, and ellipse fitting is performed on the segmented light spot image.

Step S404: If the segmented light spot image can be ellipse-fitted, step S405 is performed and an approximate depth distance of a cutting starting position and/or a cutting ending position is calculated according to a fitted curve, so as to acquire its position coordinate information.

If the segmented light spot image cannot be ellipse-fitted, an endoscopic apparatus is moved, the position is reselected, and steps S401 to S406 are repeatedly performed until the position information can be acquired.

A position at which the urethral orifice is observed through the bladder neck by the endoscope or an equivalent position that is reached as determined by the processor is used as the cutting starting position. A position at which the endoscope moves to near the seminal colliculus or an equivalent position that is reached as determined by the processor is used as the cutting ending position.

In this embodiment, the light spot information can be identified by neural networks and other image algorithms to assist in acquiring the depth information of the target points, and at the same time, in combination with the method of manual observation to ultimately determine the coordinate information of the cutting starting position and the cutting ending position. If necessary, it may also be used to determine the coordinate information of other sensitive positions and the position of the target points, which is fed back to the processor for analyzing and processing.

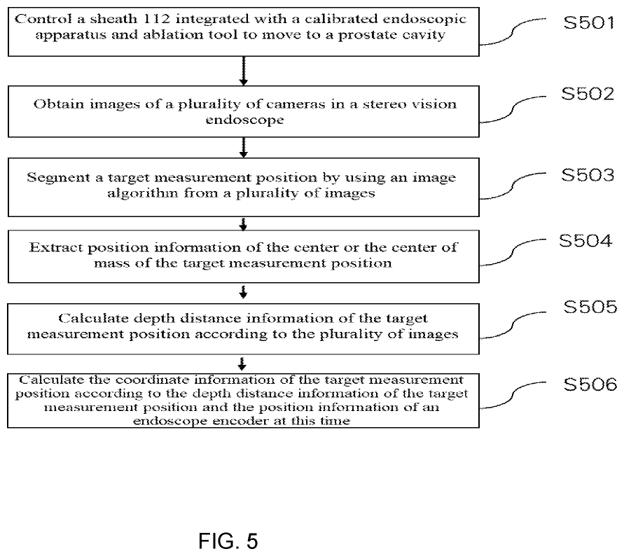

In some other embodiments, a stereo vision endoscope is employed to acquire the target position information, and an acquisition method 500, as shown in FIG. 5, includes the following specific steps.

Step S501: An endoscopic apparatus, an ablation tool and a sheath 112 are calibrated, the sheath 112 integrated with the calibrated endoscopic apparatus and ablation tool is inserted into a urethra, and a processor is used to control a first mechanical arm 110, so that the sheath 112 integrated with endoscopic apparatus and the ablation tool moves to a preset position of a prostate cavity.

Step S502: The stereo vision endoscope contains a plurality of cameras, and the plurality of cameras simultaneously photograph a target measurement position (such as a laser light spot position or a sensitive position) to obtain a plurality of images. During the process of acquiring the coordinate information of the cutting starting position and the cutting ending position, the target measurement position is a position where the urethral orifice is observed through the bladder neck by the endoscope or an equivalent position that is reached as determined by the processor, which is used as the cutting starting position. A position at which the endoscope moves to near the seminal colliculus or an equivalent position that is reached as determined by the processor is used as the cutting ending position.

Step S503: For the plurality of acquired images, the target measurement position is segmented by using an image algorithm, etc.

Step S504: The processor extracts the position coordinate information of the center or the center of mass of the target measurement position, which is fed back to the processor.

Step S505: Depth distance information of the target measurement position is calculated by the processor according to the information of the plurality of acquired images in step S502 and the acquired position coordinate information of the center or the center of mass of the target measurement position in step S504.

Step S506: Coordinate information of the target measurement position is calculated by the processor according to the acquired depth distance information of the target measurement position in step S505 and the position information of the endoscope encoder at this time and recorded.

Those skilled in the art can understand that the embodiments of this application may be provided as a method, a system, or a computer program product. Therefore, the present invention may use a form of hardware-only embodiments, software-only embodiments, or embodiments combining software and hardware. Moreover, the present invention may use a form of a computer program product that is implemented on one or more computer-usable storage mediums (including but not limited to a disk memory, a CD-ROM, an optical memory, and the like) that include computer-usable program code.

This application further provides a computer-readable storage medium storing a computer program thereon, the program, when executed by a processor, implementing the method according to any one of the embodiments of this application.

Further, this application further provides an electronic device, including a memory, a processor, and a computer program that is stored in the memory and can be run in the processor, wherein the processor implements the method according to any one of the embodiments of this application when executing the computer program.

The present invention is described with reference to flowcharts and/or block diagrams of methods, devices (systems), and computer program products according to embodiments of the present invention. It is to be understood that computer program instructions can implement each procedure and/or block in the flowcharts and/or block diagrams and a combination of procedures and/or blocks in the flowcharts and/or block diagrams. These computer program instructions may be provided to a general-purpose computer, a special-purpose computer, an embedded processor, or a processor of another programmable data processing device to generate a machine, so that an apparatus configured to implement functions specified in one or more procedures in the flowcharts and/or one or more blocks in the block diagrams is generated by using instructions executed by the computer or the processor of another programmable data processing device.

These computer program instructions may alternatively be stored in a computer-readable memory that can instruct a computer or another programmable data processing device to operate in a specific manner, so that the instructions stored in the computer-readable memory generate an artifact that includes an instruction apparatus. The instruction apparatus implements a specific function in one or more procedures in the flowcharts and/or in one or more blocks in the block diagrams.

An memory may include a form of a volatile memory, a random access memory (RAM) and or a non-volatile memory in the computer-readable medium, such as a read-only memory (ROM) or a flash memory (flash RAM). The memory is an example of the computer-readable medium.

It should also be noted that, the terms “comprise”, “contain” or any other variation thereof, are intended to cover a non-exclusive inclusion, such that a process, method, commodity or device that includes a series of elements does not include only those elements but include other elements not expressly listed or include elements inherent to such a process, method, commodity, or device. Without more limitation, the elements limited by the statement “including one . . . ” do not exclude the presence of other identical elements in the process, method, commodity, or device that includes the elements. The terms cited in this application, such as “front”, “back”, “forward”, “backward”, and the like, are only for the sake of clarity and are not intended to limit the scope of implementation of this application, and any changes or adjustments in their relative relationships, without substantial changes in technical content, shall also be considered as falling within the scope of implementation of this application.

The above embodiments of this application have been described in detail, but the described content is only the preferred embodiments of this application, and cannot be considered for limiting the range of implementation in this application. Any equal changes and improvements, etc. made within the invention scope of the present application shall still fall within the patent coverage of the present application.

1. A multi-image information fusion system for tissue cutting path planning, comprising:
a motion control apparatus, wherein the motion control apparatus comprises a fixed reference component, and a first motion control component and a second motion control component that are connected to the fixed reference component;
an ablation tool module, wherein the ablation tool module comprises an ablation tool and an endoscope with a position feedback apparatus, the ablation tool module can acquire position information of a target tissue working area, and both the endoscope and the ablation tool are connected to the first motion control component;
a three-dimensional ultrasound imaging module, wherein the three-dimensional ultrasound module comprises an ultrasonic probe, the ultrasonic probe can be used for acquiring three-dimensional ultrasound image information of a target tissue, and the ultrasonic probe is connected to the second motion control component; and
a processor, wherein the processor can extract two-dimensional slice image contour position information of the three-dimensional ultrasound image, and fuse the acquired position information of the target tissue working area and the two-dimensional slice image contour position information of the target tissue into the same standard coordinate system.

2. The multi-image information fusion system for tissue cutting path planning according to claim 1, characterized in that, both the endoscope and the ablation tool are connected to the first motion control component through a first adapter; and the ultrasonic probe is connected to the second motion control component through a second adapter.

3. The multi-image information fusion system for tissue cutting path planning according to claim 1, characterized in that, the first motion control component and/or the second motion control component is a mechanical arm or a support provided with a position feedback apparatus.

4. The multi-image information fusion system for tissue cutting path planning according to claim 1, characterized in that, the position feedback apparatus is an encoder.

5. The multi-image information fusion system for tissue cutting path planning according to claim 1, characterized in that, the standard coordinate system is a coordinate system where the fixed reference component is located.
